(19) United States
(12) Patent Application Publication
(10) Pub. No.: US 2016/0187311 A1
(43) Pub. Date: Jun. 30, 2016

(54) Respirometer

(71) Applicant: BACTEST LIMITED, Cambridgeshire (GB)

(72) Inventors: Annie Brooking, Cambridge (GB); Derek Price, Cambridge (GB)

(21) Appl. No.: 14/910,290

(22) PCT Filed: Aug. 6, 2014
(86) PCT No.: PCT/GB2014/052406

§ 371 Date: Feb. 5, 2016

(30) Foreign Application Priority Data
Aug. 6, 2013 (GB) 1314059.5
May 28, 2014 (GB) 1409407.2

(51) Int. Cl. G01N 33/18

(52) U.S. Cl. CPC G01N 33/18; G01N 33/1806

(57) ABSTRACT

We describe a floating respirometer, in particular for monitoring an activated sludge vessel of a sewage treatment plant. The device comprises: a buoyancy device to allow the respirometer to float in an aqueous liquid; a respirometer chamber, supported by the buoyancy device and arranged such that, when the respirometer is floating in said aqueous liquid, said chamber is partially filled with said aqueous liquid and defines an enclosed head space above said aqueous liquid; and a gas sensor in gaseous communication with said head space.

### FIELD OF THE INVENTION

This invention relates to sensing devices and methods, in particular for monitoring living organisms in water, in some preferred applications for monitoring activated sludge in sewage treatment plants.

### BACKGROUND TO THE INVENTION

Waste water treatment accounts for a surprisingly large proportion of the total UK energy supply, by some estimates up to 2%. The majority of this energy goes to aeration of the biological floc in a treatment plant. By way of an example, a sewage treatment plant might have perhaps twenty 100KW motors running continuously to provide aeration for the activated (bacteria-containing) sludge. It is likely that a much lower degree of aeration would suffice, but understandably a plant manager will err on the side of caution. The process of keeping a waste water treatment plant running satisfactorily is relatively poorly understood and generally not closely controlled.

In the main sight and smell are used by experienced managers to control a plant (when operating properly, the smell is not unpleasant), supplemented by occasional tests. In the UK typically the BOD5 test (biochemical oxygen demand 5 day test) is generally used which, as the name implies, incubates a field sample over five days to characterise the sample by its oxygen use. Sometimes probes such as an oxygen or ammonia probe are also employed although in practice these do not work well and often fail or go out of calibration.

Better understanding and control of the process could enable substantial energy savings by reducing the degree of aeration to just that which is necessary. However it is difficult to obtain accurate measurements of the status of a plant, and in particular of the biological oxygen demand of the activated sludge. This is in part because the aqueous sludge is not a pure liquid but includes insoluble particles, household/industrial rubbish and the like.

### SUMMARY OF THE INVENTION

According to the present invention there is therefore provided a floating respirometer, comprising: a buoyancy device to allow the respirometer to float in an aqueous liquid; a respirometer chamber, supported by the buoyancy device and arranged such that, when the respirometer is floating in said aqueous liquid, said chamber is partially filled with said aqueous liquid and defines an enclosed headspace above said aqueous liquid; and a gas sensor in gaseous communication with said headspace.

There are a number of problems which employing a floating respirometer of this type addresses: as described later, for consistent results it is important that the volume of liquid sample within the chamber is substantially constant from one sample to the next because the volume within the chamber effects the signal, but this is difficult to achieve with a fluid containing high solids content which cannot easily be pumped.

Floating the respirometer provides a natural, repeatable control of the volume of fluid within the chamber since the fluid rises within the chamber to the level of the external air-liquid interface. The volume may be controlled by controlling the size of the chamber and the overall buoyancy of the device. The buoyancy may be provided by one or more floats or the buoyancy device may be inbuilt, for example as a second, air-filled chamber and/or by mounting the sample chamber on a buoyant platform. The skilled person will recognise that many different types of buoyancy arrangement may be employed.

In some preferred embodiments the gas sensor is a gas pressure sensor, more particularly of the type employing a flexible diaphragm or membrane so that the head space of the chamber remains sealed, but additionally or alternatively gas composition sensors may also be employed, for example to sense a level of oxygen, nitrogen, ammonia, carbon dioxide or the like. In preferred embodiments, in particular where pressure is measured, the chamber is sealed during a measurement of respiration of living organisms in the liquid within the chamber, but in principle this is not essential for other types of measurement.

A further advantage of employing a floating respirometer is that of temperature control—the liquid sample within the chamber is at, and remains at, the temperature of the surrounding liquid. For a floating respirometer monitoring an activated sludge vessel this means that the respiration measurement will be an indication of the actual biochemical oxygen demand of the activated sludge because it is at the same temperature as the sludge for which, in embodiments, the degree of aeration is to be controlled. Further, because the temperature of activated sludge does not change significantly over a period of hours, but rather month-to-month, a floating arrangement ensures a steady, substantially constant temperature from one measurement to the next. This, again, is particularly important when measuring changes in gas pressure due to respiration of living organisms within the liquid in the chamber because the observed pressure changes may be very small, for example or order 0.1 millibar, and thus temperature control is important to avoid false readings caused by temperature changes.

In some embodiments the respirometer may be substantially free-floating, for example supported by a flotation ring and tethered, for example by an umbilical providing power and/or compressed air. In other arrangements the device may float attached to an arm, arranged so that the respirometer can float up and down as necessary. In embodiments the culture chamber may be generally cylindrical; larger volumes are generally preferable, for example greater than one litre. The chamber need not have the same cross-sectional area at all depths when floating. It will be appreciated that if the chamber has a small cross-sectional area at the level of the air-water interface then the head space volume will be relatively small and also a small change in the depth at which the device floats will result in a small change in the volume of liquid within the chamber (conversely a large cross-sectional area at the air-water interface will make the chamber to liquid volume more sensitive to flotation height). Thus the cross-sectional area of the chamber at the height of the air-liquid interface may be adjusted to adjust the sensitivity of the device to changes in buoyancy and, optionally, the area at this height may be less than the cross-sectional area at a deeper part of the chamber, for increased volume control accuracy.

Nonetheless, in embodiments the culture vessel chamber may be generally tubular and the majority of the chamber may hang below a floating platform on which the chamber is mounted. In pressure-measuring embodiments the ratio of the head space volume to the liquid phase volume within the chamber affects the rate of pressure change and, whatever the shape of the chamber, this is automatically regulated by the buoyancy of the device (and may be adjusted by adjusting the buoyancy).

As previously mentioned, another problem especially when monitoring difficult to pump liquids such as activated sludge, is how to obtain an accurate volume of sample. In preferred embodiments of the device an inlet valve is provided beneath the level at which the chamber floats so that the chamber can be filled by opening this valve. In preferred embodiments this valve is at a lower end of the chamber, for example in a base of the chamber. Then the liquid in the chamber may be expelled by pumping air into the chamber. Conveniently this may be achieved by re-using a mechanism to promote gaseous exchange between the liquid and head space within the chamber, more particularly an air sparge arrangement as described later. In preferred embodiments the inlet valve comprises a pinch valve, in particular a length of tubing which may be closed by pinching sides of the tubing together, for example by compressing the tubing with compressed air. Such an arrangement is advantageous since it is able to seal around solid particles which have passed partway through the length of the valve.

In some preferred embodiments, in particular for use in an activated sludge vessel, the respirometer includes a bubble shield beneath the inlet valve. This may comprise a grid beneath the lower end of the chamber, the grid having a surface extending upwards from beneath the respirometer outwards, towards, and preferably beyond, the sides of the respirometer to divert bubbles from beneath away from the respirometer. This arrangement is particularly useful for an activated sludge vessel in which there may be aeration of the vessel from beneath, such a shield inhibiting bubbles from entering the sample chamber. Advantageously holes in the grid may also be sized to provide a filtering effect to inhibit larger unwanted entities from entering the inlet valve.

Especially where pressure changes are being measured, because the changes in pressure from respiration are very small it is important to ensure adequate gaseous exchange between gasses within the liquid sample in the chamber and the headspace. More particularly in embodiments there should be a system which promotes such gaseous exchange to a sufficient degree that this occurs faster than a rate of gas use/production by the living organisms in the liquid sample—in essence gas must be exchanged between the liquid and head space faster than the organisms use the gas. This is preferable because otherwise the gas pressure change signal could be misleading, since absent sufficient mixing between the liquid in the chamber and the gas in the headspace the pressure measurement could be limited by the rate of gaseous exchange between these. The required degree of mixing can be established experimentally for particular conditions by measuring respiration (pressure change) of a sample and reducing the degree of mixing until it can be seen that pressure changes are dependent on (limited by) the mixing rate—this establishes a lower limit for the mixing rate.

Preferred embodiments of the floating respirometer, therefore, include a mixing system to promote gaseous exchange between the liquid in the chamber and the gas in the head space of the chamber. Various techniques may be envisaged, for example a paddle wheel or a pumping arrangement. As previously mentioned, In preferred embodiments the gaseous exchange is very fast, in particular to move oxygen from the head space into the liquid, reducing the partial pressure or oxygen in the head space. One approach is to employ a pump in combination with a venturi arrangement in the head space, but as previously mentioned, pumps have drawbacks for activated sludge samples. A preferred approach, which has been found to work well in practice, is to employ a sparge to bubble gas from the head space through the liquid in the chamber (in a sealed system). Thus in embodiments an air pump may be provided to pump gas from the head space along a conduit into the liquid in the chamber, preferably to pump the gas down to the bottom of the chamber, for example to a sparge ring, to allow the gas to bubble up through the liquid back to the head space.

In preferred embodiments the respirometer is provided with at least one gas/air valve in communication with the head space of the chamber. In this way the chamber can be filled by opening the inlet valve at the bottom of the chamber and the gas (outlet) valve at the top of the chamber, and the device can be purged by closing the gas valve at the top of the chamber and providing an external air supply into the chamber, for example re-using the sparge system, to blow air into the chamber to expel liquid in the chamber out through the inlet valve at the bottom. (It will be appreciated that even when blowing air in at the bottom of the chamber, this air will rise to the top and push the liquid out through the inlet valve at the bottom). In embodiments there may be two gas/air valves provided for the device, an air release valve in communication with the head space, to allow air out of the head space as liquid fills the chamber, and an air inlet valve to allow air into the chamber, for example into the sparge system, to expel the liquid to purge the chamber of liquid. In embodiments the respirometer is provided with a controller to appropriately sequence these valves (air inlet open, air outlet closed, liquid inlet open to purge the device; air inlet closed, air outlet open, liquid inlet open to fill the device; liquid inlet closed, air inlet closed, air outlet closed to operate the device as a respirometer).

In some preferred embodiments the valves are operated by a pneumatic control system; conveniently in embodiments the compressed air operating the valves may also be employed to drive the sparge system, more particularly a gas recirculation pump of the sparge system. The gas (pressure and/or composition) sensor may be battery powered or powered by a low voltage supply forming part of an umbilical connection for the device also providing the compressed air. Gas sensing signals from the device may either be provided wirelessly or via a wire connection again, for example, via the umbilical.

In other aspects of the invention a respirometer is provided which is not necessarily floating but which incorporates one or more of the previously described features of sample inlet/outlet control and/or gas sensing.

As previously mentioned, some particularly preferred applications of a floating respirometer as described above are in monitoring an activated sludge processing plant and more particularly the floating respirometer may provide a signal which can be used to control a degree of aeration of an activated sludge vessel of the plant, either manually or automatically. Broadly speaking a pressure drop measured by a pressure sensor in the headspace correlates with a degree of biochemical oxygen demand of the living organisms in the activated sludge, and thus knowing this demand the degree or aeration can be adjusted accordingly. The skilled person will appreciate it is not necessary to know an absolute value—a signal indicating a relative increase or decrease of biochemical oxygen demand can be employed to correspondingly increase or decrease a degree of aeration of the plant. In more sophisticated approaches, a rate of change of pressure may be employed additionally or alternatively to a change (reduction) in pressure measurement for controlling the degree of aeration.

Although the device is particularly useful for monitoring an activated sludge vessel, it will be appreciated that there are other applications for an accurate, sensitive floating respirometer of this type and, in general, the respirometer may be employed to monitor and/or control living organisms in any water-based production or processing stage of an industrial plant. For example another useful application of the device is in monitoring a degree of contamination of water-based paint by living organisms.

Thus in a related aspect the invention provides a method of monitoring a production plant such as a water-based paint production plant, by employing a floating respirometer, in particular as described above, to identify the presence and/or quantity of living organisms in a water-based liquid in the plant, in particular to provide a signal indicative of contamination of the water based liquid, or paint from the plant, by living organisms.

A measurement of respiration by the device may take, for example, a period of order of one hour or longer. Broadly speaking in operation the device is flushed or purged, refilled, sealed, and observed for a period of time, for example, half an hour, one hour or longer, measuring a pressure change, more particularly a pressure reduction, as bacteria and/or other living organisms use gas in particular oxygen, during respiration. Depending upon the number of organisms, quantity of food, desired sensitivity of measurement and the like the measurement period may be extended to a few hours. There may be applications in which more frequent measurements are desired. In this case a set of floating respirometers may be employed, synchronised to pull and close at intervals (of less than the measurement interval) so that after an initial start-up period successive devices provide successive measurements at intervals which are less than the duration of a single measurement.

In general, embodiments of the respirometer/method may be employed to measure both positive and negative pressure changes. For example, depending upon the process monitored gases such as carbon dioxide and/or nitrogen may be produced as part of the microbial respiration process. For example carbon dioxide may be monitored in a fermentation process.

Additionally or alternatively a plurality of floating respirometers may be employed in a plant. For example floating respirometers may be positioned at intervals along the length of an activated sludge vessel in a direction of flow of liquid through the vessel. This is because the conditions change with distance along the flow direction in a sludge vessel (where the liquid may take several hours to transit), for example using more oxygen at the start and more nitrogen at the end. Thus a potentially more accurate determination of the operating condition of a plant may be achieved using a set of sensors and, optionally, the degree of aeration at different locations within an activated sludge vessel may be different depending upon the locally determined conditions of operation as established local to a respective set of floating respirometers.

In a related aspect the invention provides a method of measuring a degree of respiration of living organisms in an aqueous medium, the method comprising: providing a respiration measuring device in said aqueous medium, in particular floating said respiration measuring device in said aqueous medium, said respiration measuring device comprising a respiration chamber; enabling said respiration measuring device, in particular said respiration chamber, to partially fill with said aqueous medium, leaving a headspace above the aqueous medium within the device; allowing said living organisms to respire within said respiration measuring device, in particular within said respiration chamber, such that a gaseous pressure or composition of said headspace is altered; and measuring said alteration in said gaseous pressure or composition of said headspace to measure said degree of respiration of said living organisms.

In a further aspect the invention provides a floating respirometer, in embodiments for sewage monitoring.

### Biomass Retention

In a respirometer as previously described, whether or not the respirometer is floating, an alternative form of monitoring may be employed in which the biomass is deliberately retained in the respirometer from one sample to the next. Generally this is considered undesirable and precautions are taken to prevent this, for example by coating internal surfaces of the respirometer with PTFE. However by retaining biomass within the respirometer a high concentration of biomass can be achieved. More particularly some waste water processing systems employ immobilised biomass, capturing and retaining the biomass on a solid matrix rather than allowing the biomass to remain suspended in the waste water. In this way, a high concentration of biomass can be retained within a treatment process vessel so that the vessel can be smaller and has a reduced need for settlement of final solids. Broadly speaking the solid matrix becomes colonised by a mixed biomass micro-flora which is then retained within the process vessel rather than having to be returned to it in the Return Activated Sludge to seed the process. However this poses a difficulty for a respirometer which measures, for example, biochemical oxygen demand or food to biomass ratio, since there may be very little biomass in a waste water sample, in particular at the exit end of the processing system.

In a further aspect the invention therefore provides a respirometer comprising: a sample inlet; a sample outlet; a respirometer chamber to contain an aqueous liquid sample; a gas sensor to sense a pressure and/or composition of gas in said respirometer chamber; wherein said respirometer chamber further comprises a biomass growth support region or matrix.

By providing the respirometer chamber with a biomass growth support region, or solid matrix, a colony of micro-flora can be built up and retained within the respirometer chamber which matches that in the waste water processing system.

In one embodiment the biomass growth support region comprises one or more curtains of material, for example fibrous or woven polymer material akin to polymer “towelling”. Additionally or alternatively polymer beads, granules or pellets may be provided within the respirometer chamber to increase the internal surface area. More generally one or more regions or walls of the respirometer chamber may be spongy or sufficiently roughened to retain biomass (ie. having a surface fractal dimension of greater than 2).

One or more features of the previously described respirometer embodiments may be incorporated. Thus for example in embodiments the sample outlet is at a lower end of the sample chamber and a gas or air inlet/supply is provided to purge the respirometer, pushing the fluid down and out through the outlet before refilling. In particular where beads or pellets are employed a mechanical filter or other means may be provided to retain the granules within the respirometer.

The invention also provides a method of monitoring an aqueous medium, in particular an aqueous medium in a waste water treatment plant, by measuring a degree of respiration of living organisms in an aqueous medium, the method comprising: providing a respiration measuring device in said aqueous medium, said respiration measuring device comprising a respirometer chamber; enabling said respirometer chamber to partially fill with said aqueous medium, leaving a headspace above the aqueous medium within the device; allowing said living organisms to respire within said respirometer chamber such that a gaseous pressure or composition of said headspace is altered; and measuring said alteration in said gaseous pressure or composition of said headspace to measure said degree of respiration of said living organisms; the method further comprising: repeating said measuring on successive samples of said aqueous medium whilst returning said living organisms within said respirometer chamber from one said sample to the next.

As previously mentioned, such a technique is particularly advantageous for monitoring an exit flow of a waste water treatment plant, in particular a plant with immobilised biomass, since in such a plant there may be very little biomass in the exit fluid.

In embodiments the above described method may be employed (anywhere within the fluid flow) to monitor a colonisation profile of the plant. Preferably the biomass growth support region (solid matrix) employed is of the same type as used in the plant, and when fresh matrix is added to the plant/respirometer successive measurements can monitor colonisation of the matrix.

### Closed Loop Control

We have previously described, in our co-pending unpublished patent application GB1214563.7, a method of (and system for) closed-loop control of a waste water treatment plant, the method (system) comprising (means for): obtaining a fluid sample from a fluid of said plant; providing said fluid sample to a sealed chamber such that said fluid sample incompletely fills said sealed chamber leaving a headspace; incubating said fluid sample in said sealed chamber; determining a change in pressure in said headspace during said incubating; and controlling a degree of aeration of said waste water treatment plant responsive to said change in pressure.

Embodiments of this method/system may employ a floating respirometer to monitor the waste water, more particularly an activated sludge vessel of the plant and/or one or both of influent to the plant and returned activated sludge for the plant.

Thus in a further aspect the invention provides a method of closed-loop control of a waste water treatment plant, the method comprising: sampling a fluid in said plant using a floating respirometer by providing said fluid sample to a chamber of the respirometer such that said fluid sample incompletely fills said chamber leaving a headspace; sealing the chamber; incubating said fluid sample in said sealed chamber; determining a change in gas pressure or composition in said headspace during or after said incubating; and controlling a degree of aeration of said waste water treatment plant responsive to said change in gas pressure or composition.

The change, more particularly drop, in pressure in the headspace of a sealable chamber of the floating respirometer may be employed to monitor one or more of an activate sludge vessel (at one or more locations), influent, and RAS (returned activated sludge) in a waste water treatment plant. The change in pressure is believed to result from a combination of use of some gasses, in particular oxygen, in growing bacteria and production of other gasses such as carbon dioxide, during respiration/bacterial growth. Experimentally an initial pressure drop is observed over a period up to one to a few hours followed by a flattening of the curve and subsequent rise in pressure. The initial drop in pressure has been observed experimentally to correlate with the food available to the bacteria and other organisms in the sample, and with the biomass in the sample. More particularly the observed pressure change is believed to correlate with the biochemical oxygen demand (BOD) of the fluid sample. It has further been established that measurements in one or more of these locations in a sewage treatment plant may be employed in closed-loop control of the plant, more particularly the aeration, potentially with a corresponding loop time of less than 8, 4, 2 or 1 hours.

Controlling the aeration in this manner enables the method (and the corresponding system) to determine a sufficient level of aeration without wasting energy in excess aeration, at the same time ensuring that the clear output from the waste water treatment plant has sufficiently low BOD for this to be safely discharged into a water course. The control may be responsive to, for example, one or more of a pressure drop, a rate of pressure drop (for example pressure drop per hour), and an integrated pressure drop (area under a pressure-time curve) as measured by the floating respirometer. Additionally or alternatively the control may be responsive to one or more of: a measured level (or partial pressure) of, and/or rate of change of level of, and/or integrated change of, one or more gases in the headspace. Such gases may comprise, for example, one or more of: oxygen, nitrogen, ammonia, and carbon dioxide. In embodiments multiple sensors and/or chambers may be provided to enable multiple signals to be averaged for more accurate measurement.

As previously mentioned, optionally a floating respirometer may sample the influent to the plant, in effect to measure the level of food in the influent; and/or a floating respirometer may sample the RAS, in effect to measure the quantity of living biological material (biomass) in the plant. The degree of aeration may then be controlled responsive to a combination of these parameters, for example a ratio of food to biomass (although in principle some other combination may be employed, for example subtracting one parameter from the other). In a simpler approach, however, the degree of aeration in an activated sludge vessel may be determined by a measurement made by the floating respirometer in the vessel (for example a pressure measurement), which is a proxy for a measurement of the BOD of the material.

Different zones of an activated sludge vessel may need different amounts of aeration, depending upon the local biology. For example oxygen may be used at one end of the vessel, where the influent enters, and proportionally more nitrogen towards the far end, where the liquid leaves. Local aeration in different regions of the vessel may be controlled by different, locally tethered floating respirometers.

The particular degree of aeration/control may be determined on a plant-by-plant basis: typically plants have their own individual characteristics and needs and the control over the aeration equipment may be adapted accordingly. In principle a plant may be categorised into one of a plurality of different sizes/profiles of plant and a starting point for a control procedure determined accordingly.

Experimentally it has been determined that varying the sample to headspace ratio can significantly affect the observed change in pressure and can be used as a mechanism to adjust the sensitivity of the measurement (and in principle, the loop gain of the control loop). This ratio may be controlled by adjusting the height at which the respirometer floats, for example by adjusting the buoyancy.

In a related aspect the invention provides a system for closed-loop control of a waste water treatment plant, the system comprising a floating respirometer for sampling a fluid in said plant, said respirometer having a sealable chamber, wherein said fluid sample incompletely fills said chamber leaving a headspace; a system for sealing the chamber and incubating said fluid sample in said sealed chamber; a sensor to determine a change in gas pressure or composition in said headspace during or after said incubating; and a system to provide a control signal for controlling a degree of aeration of said waste water treatment plant responsive to said change in gas pressure or composition.

The control signal may be provided to a data processing system, for automatic control of the plant, more particularly the aeration, or the signal may be provided, for example on a screen or printout to a user for manual adjustment/control of the aeration system.

### BRIEF DESCRIPTION OF THE DRAWINGS

These and other aspects of the invention will now be further described, by way of example only, with reference to the accompanying figures in which:

FIGS. 1a and 1b show, respectively, a high level schematic diagram of a waste water treatment plant, and schematic block diagram of a control system for closed-loop control of a waste water treatment plant;

FIGS. 2a and 2b show a culture vessel which may be adapted for use in embodiments of the invention, showing the vessel under, respectively, normal atmospheric pressure and reduced pressure;

FIG. 3 shows the variation of pressure with time when incubating influent over a period of hours;

FIG. 4 shows a floating respirometer according to an embodiment of the invention;

FIG. 5 shows a floating respirometer according to an alternative embodiment of the invention;

FIG. 6 shows a sewage treatment plant control system according to an embodiment of the invention; and

FIGS. 7a to 7c show a respirometer with biomass retention according to an embodiment of the invention, a tethered floating respirometer, and the use of multiple floating respirometers in a waste water treatment plant with segmented aeration.

### DETAILED DESCRIPTION OF PREFERRED EMBODIMENTS

### Activated Sludge Monitoring

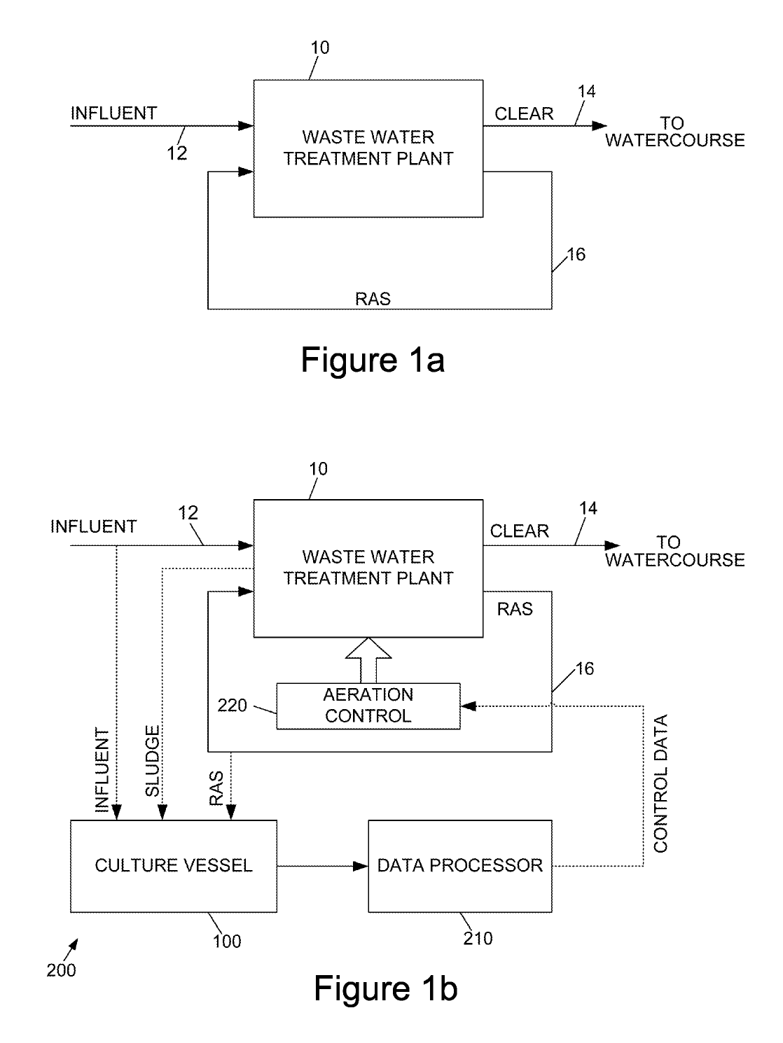

FIG. 1a shows, at a high level, a schematic diagram of the operation of a waste water treatment plant 10. Thus the plant accepts influent 12, fluid from which the solids have been substantially removed, containing a high level of ‘food’ for bacteria, protozoans rotifers, fungi and the like (biomass′) and having a high biochemical oxygen demand (BOD). The output from the plant has two components, a clear component 14 which may be provided to a water course and a biological component 16 comprising living biological material referred to as returned activated sludge (RAS), typically at around 60% concentration. The RAS is provided back to the input side of the plant to help maintain the eco system.

FIG. 1b shows a block diagram of a closed loop based water treatment control system 200 to implement real time closed loop control of a sewage treatment plant based on a pressure and/or composition measurement of the gases in the headspace of a closed vessel/sealed chamber. Thus one or more of influent (“food”), sludge from the sludge vessel, and RAS samples are provided to a culture vessel, and the overall changes in gas pressure/composition are monitored by data processor 210, for example a general purpose computer under software control. The data processor may output one or more parameters indicating the BOD at one or more locations in the system, for example on a screen for an operator to use in controlling the plant or to an aeration control system 220 to automatically control the aeration such that it is sufficient, but not significantly in excess of that required given the amount of food/biomass the plant is coping with. This in turn enables the plant to operate efficiently but also to react to shock loads and variations in food/biomass levels over time periods of one or more days, weeks, months or years.

We have previously described a system for monitoring the metabolism/growth of microorganisms, the system comprising a sealed chamber with a flexible diaphragm to provide sensitive pressure measurements of gas pressure in the headspace above a culture liquid. For details reference may be made, for example, to our U.S. Pat. No. 8,389,274.

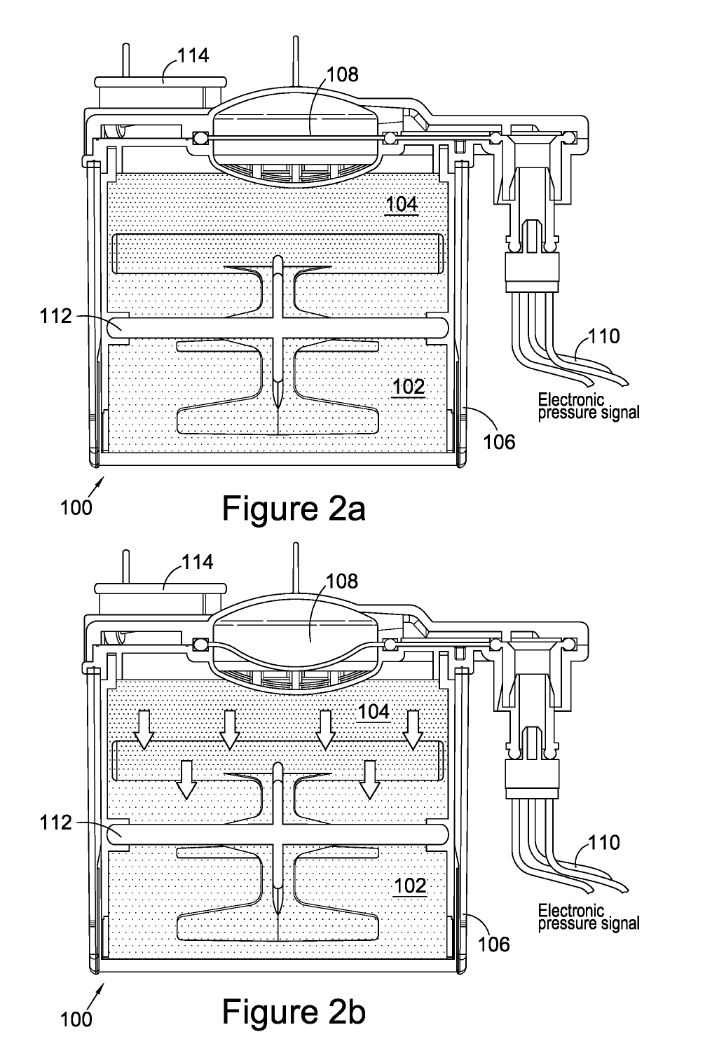

It is helpful to outline details of such a device since a similar pressure measuring system may be adapted for inclusion in embodiments of the invention described later. Thus FIGS. 2a and 2b show, schematically, an embodiment of a similar device 100 to that in U.S. Pat. No. 8,389,274 under, respectively, normal atmospheric pressure and negative pressure (in operation either negative pressure or positive pressure may be produced). Thus a culture 102 of biological material undergoes metabolism and growth during which it exchanges gases with the aqueous liquid (water) carrying cells depending upon various factors gas may be used and/or produced, for example the cells may produce carbon dioxide during respiration. A gaseous headspace 104 of the sealed culture chamber 106 thus experiences changes in pressure due to exchange of gas with the culture medium, and these are monitored by a diaphragm 108 and converted to an electronic pressure signal 110 which may, for example, be digitised and processed electronically by hardware, software or a combination of the two. As illustrated the device includes a sealable inlet/outlet port 114; it also includes an agitator 112, and may incorporate temperature control (not shown). The liquid phase (sample) to gaseous phase (measured head space) volume ratio can be used to adjust the sensitivity of the device—for example a ratio of up to 1:1 liquid:gas may be employed.

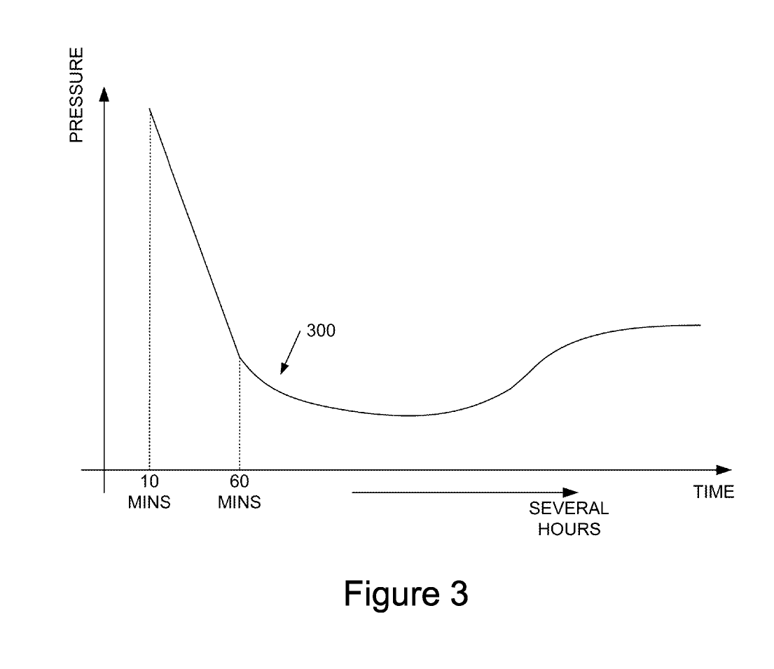

FIG. 3 shows the general shape of a pressure-time curve for a sample of liquid from a sewage treatment plant. Thus there is an initial period during which the pressure can vary and results appear unreliable. This typically lasts up to around 10 minutes. The pressure then begins to fall, flattening out in a trough region 300 after around an hour. Over a further period of several hours the pressure then gradually starts to rise once more (the graph of FIG. 3 is not to scale). The initial rate of pressure drop appears to be related to the concentration of food present, a faster drop being observed with more “food” present. (Thus either the pressure drop or the rate of pressure drop may be measured). Without wishing to be bound by theory it is surmised that the pressure drop relates to the conversion of gas into living biomass and that the trough region occurs when the oxygen has been depleted (the subsequent smaller rise relating to anaerobic respiration). In practice the pressure drop may be a measurement of both BOC and COD (chemical oxygen demand)—but if so this is potentially advantageous for aeration control.

In embodiments this approach provides a “BOD5” test proxy. More particularly the area under the pressure-time curve to this point may also be used as an indication of the amount of food available, and in embodiments may provide a better proxy for a BOD5 test.

Thus, broadly speaking, a closed vessel pressure measurement can be used as a measure of oxygen utilisation by a given body of biomass with time, consistent with the food availability. Additionally or alternatively it can be useful to control based on a food to biomass ratio. If necessary a measurement of the biomass may either be made by heating a sample, for example by microwaving the sample, to determine the dry weight of biomass or by measuring the amount of biomass indirectly by culturing the biomass.

### Floating Respirometer

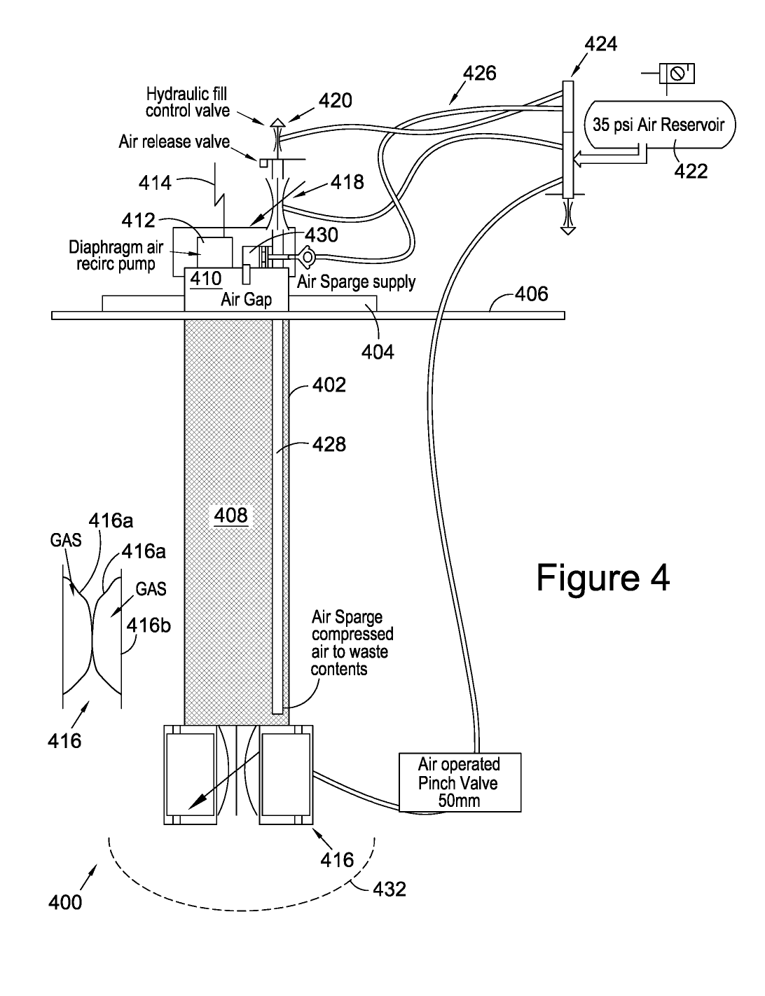

Referring now to FIG. 4, this shows a floating respirometer 400 according to an embodiment of the invention. The device comprises a chamber 402 supported on a buoyant, floating platform 404 so that the chamber straddles the air-water interface 406 (where here ‘water’ is used as shorthand for the aerated media of the activated sludge vessel). As illustrated the chamber is filled with the aqueous activated sludge medium 408 up to the level of the air-water interface, leaving an air gap 410 in an upper, headspace region of the chamber. One or more diaphragm-based pressure sensors 412, of the general type illustrated in FIG. 2, measure the air (gas) pressure in this headspace. In one embodiment four sensors are used and the outputs averaged for increased accuracy. As illustrated schematically by antenna 414, the sensor(s) may have a wireless communication link with an on-shore data processor/controller to interpret the data from the sensor(s) to provide one or more of pressure change data, BOD data and oxygen demand/aeration control data. The on-shore controller (not shown) may also control a compressed air system to operate valves to fill and empty the device and to operate the air sparge supply, as described further later. It will be appreciated that the sensor link may be wired or wireless and the sensors may be battery powered or powered by an external connection.

At the bottom of the chamber there is an air-operated pinch valve 416, also schematically illustrated in the inset, comprising a rubber sleeve 416a which can be compressed on its length by pressurised gas between the sleeve and a surrounding cylindrical wall 416b of the valve. As can be seen from the inset, the sleeve is capable of sealing whilst having particles trapped between the walls of the sleeve. At the top of the chamber, in gaseous communication with the headspace 410, a pair of valves 418, 420 is provided; these may but need not be pinch valves. Valve 418 is an air release valve, operable to allow air within the chamber 402 to escape as the chamber fills from the bottom. Valve 420 is a fill control valve, operable to provide pressurised air into the chamber, for example via the air sparge supply.

These valves are driven by compressed air from a reservoir 422 via a distribution and control mechanism 424 which, in conjunction with a controller (not shown) controls a sequence operation of the valves to fill and empty the floating respirometer. Thus to empty the respirometer the pinch valve 416 is controlled open, the air release valve 418 is control shut and the fill control valve is used to pump compressed air into chamber 402, for example via the air sparge described below, thus expelling the contents of the chamber out through the pinch valve at the bottom. To fill the chamber no air is pumped in, the pinch valve at the bottom of the chamber is opened and the air release valve 418 at the top of the chamber is also opened to allow the chamber to fill driven by the hydrostatic pressure of the sludge outside. In embodiments of the floating respirometer the air supply is provided via hoses 426, which may also constitute an umbilical tethering platform 404 in a desired region of measurement.

It is important that the sludge 408 in chamber 402 is well mixed with the gas in the headspace 410 so that measurement of change of headspace pressure is not limited by the rate of gas-to-sludge mass transfer. In preferred embodiments this is achieved by an air sparge system 428 comprising a tube to carry headspace gas from the headspace to the bottom of the chamber where, optionally, the gas may be bubbled up through the chamber via a sparge ring (not shown). A pump 430 is employed to recirculate the gas; in embodiments this is driven from compressed air from reservoir 422.

In preferred embodiments a bubble shield 432 is provided beneath pinch valve 416 to divert bubbles from aeration within the sludge around the pinch valve, for better filling of the chamber 402. This shield can also serve as a mechanical filter to inhibit large solid elements from entering the chamber 402.

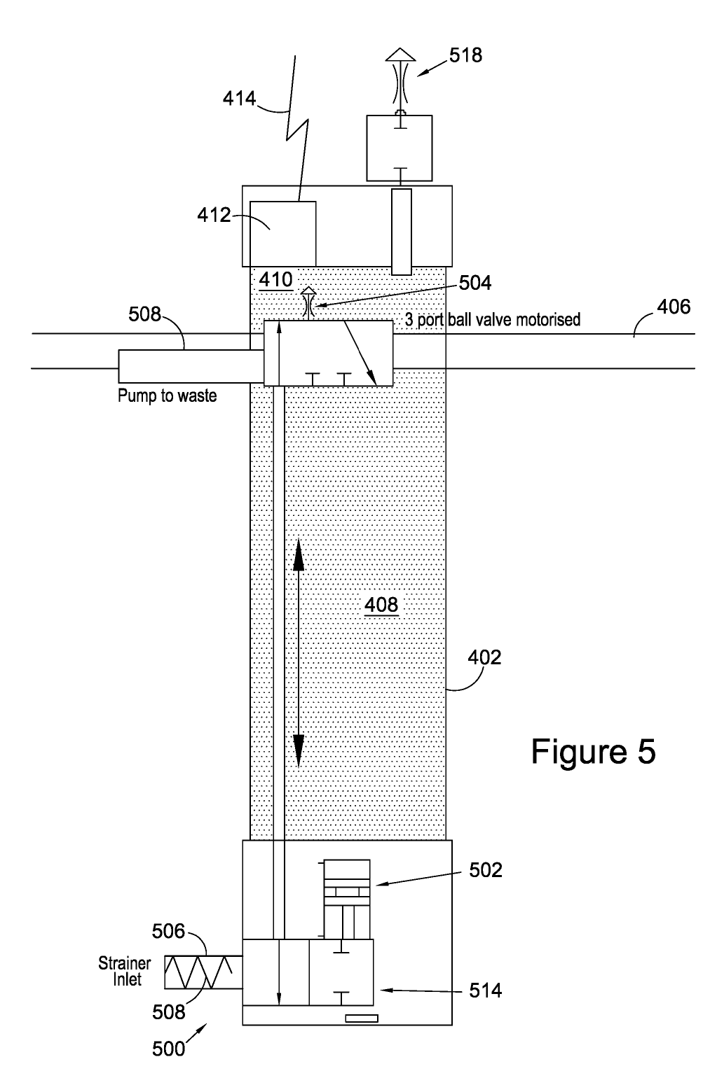

FIG. 5 shows another embodiment of a floating respirometer 500 which employs a different mechanism to fill/empty the chamber and a different mechanism to mix headspace gas with the sludge within the chamber. Thus in the arrangement of FIG. 5 a pump 502 pumps sludge from the bottom of the chamber 402 up through a Venturi device 504 located within the headspace to mix the sludge and gas. The chamber has an inlet at the bottom 506, preferably with a strainer 508 and an outlet 510 at the top. A pump may be employed to pump sludge in at the bottom and out of the top to fill/refill the chamber 402 (pump 502 may be re-used for this purpose), or a hydraulic fill arrangement may be employed as previously described. Thus, as illustrated, the device includes a controllable valve 518 operable to vent the headspace 410 of the chamber to the atmosphere; this valve may be controlled by compressed air or may be a motorised valve. In embodiments valve 518 is opened to fill chamber 402 via inlet 506 by means of hydraulic pressure; the chamber may be emptied by pump 502. In the illustrated arrangement additional valves are employed to couple the inlet and outlet with a tube 516 leading from the bottom of the chamber up to Venturi 504 forming part of the gas-sludge mixing arrangement. Thus at the bottom of the chamber a valve 514, for example a motorised L port ball valve, selectively allows sludge into the bottom of the chamber via strainer inlet 506 or allows sludge from the bottom of the chamber up through type 516 towards Venturi 504. At the top of the chamber a valve 512, which may be a 3-port motorised ball valve, selectively either couples type 516 to Venturi 504 or couples pipe 506 to waste outlet 510 so that the sludge at the bottom of the chamber may be pumped out by pump 502.

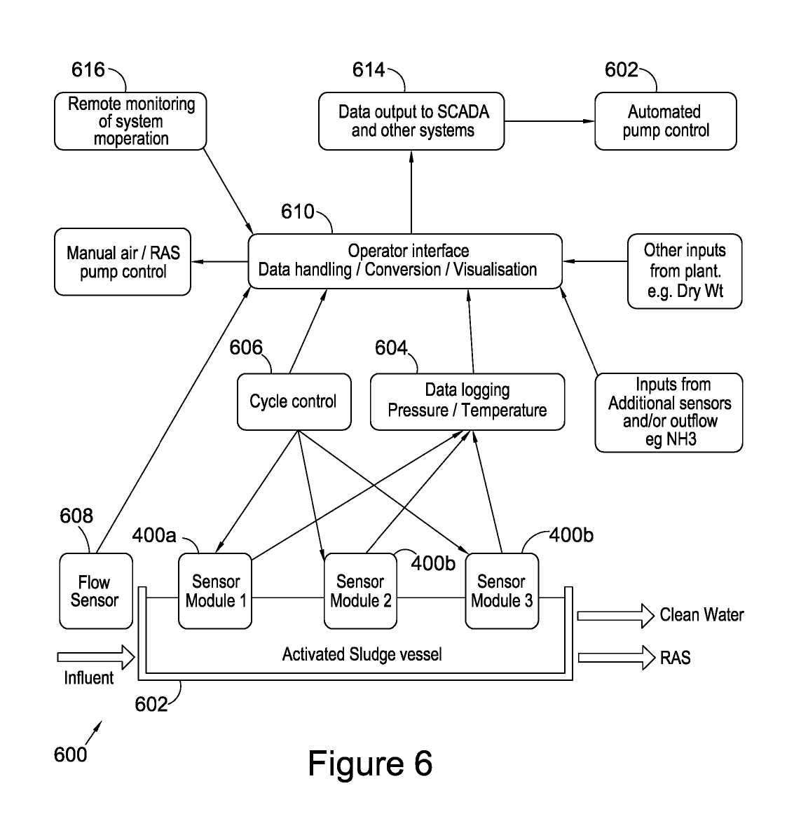

FIG. 6 shows an embodiment of a sewage treatment plant control system 600, illustrating a system of the type shown in FIG. 1 in more detail. Thus an activated sludge vessel 602 is provided (in this example) with 3 floating respirometer sensor modules 400a, b, c each coupled to a data logging system 604. In embodiments a floating respirometer may also include a temperature measuring device to provide fluid temperature data back to data logger 604. A controller 606 controls fill/measure/empty cycle operation of the floating respirometers. A flow sensor 608 measures a rate of liquid flow into and/or within activated sludge vessel 602. A data handling and visualisation system 610 is connected to the data logging system 604 to receive data from the sensor(s), to controller 606, to control when measurements are made, and to flow sensor 608. The data handling system 610 may thus receive liquid flow data and/or temperature data and/or pressure or gaseous composition measurement data from the one or more sensor modules. The data handling system 610 may present this as raw data to the operator, for example on a graphical display and/or this data may be processed, for example to convert a measurement of gaseous pressure/composition to an indication of oxygen demand and/or an indication of a need for aeration; again one or more of these may optionally be displayed graphically or output in some other manner by module 610. In general module 610 also provides an operator interface to allow control of the sensing modules to make measurements. Optionally module 610 may also receive inputs from one or more additional sensors such as an output flow rate sensor, and/or an ammonium level sensor, and the like. Module 610 may further optionally receive additional inputs from the plant, for example an input of dry biomass weight obtained as described previously from a sample of one or more locations in the vessel.

In embodiments the information output by module 610 may be employed by an operator of the plant for manual control of a level of aeration and/or for control of a flow rate of sludge through vessel 602 (by controlling a pump), and/or for controlling a degree of RAS feedback (by controlling a RAS pump). In a typical activated sludge vessel aeration may be provided by a series of tubes with holes at intervals along their length provided with an air supply and located at the bottom of the sludge vessel; these tubes may run perpendicular to the flow direction and it may be possible to control aeration so that at different locations along the flow different levels of aeration are provided. Thus the data from module 610 may be employed to control a degree of local aeration, for example in the region of a particular sensor.

Additionally or alternatively could a system 612 for automatic control of aeration/local aeration and/or of sludge flow rate and/or of RAS feedback. Optionally this control may be implemented by means of an SCADA (supervisory control and data acquisition) interface module 614. Further optionally a network connection/interface 616 may be provided for remote monitoring and/or control of the system. The skilled person will appreciate that the modules 604, 606, 610, 614 and 616 may be implemented as software modules within a computer system; the air/sludge pump control module 612 may be implemented by software with an interface to a suitable electronic controller.

In an automatic arrangement broadly speaking the system may increase a level of aeration when the oxygen demand is high as indicated by a larger measured pressure drop and vice versa. The operating region of the plant may be controlled to be different at different points along the length of flow through vessel 602—for example a region of relatively reduced oxygenation may be provided at the front end of the vessel (where the influent enters) and, for example, a quantity of nitrifying organisms may be controlled so that there is a region of increased nitrification towards an end of the flow region, optionally reducing the oxygen, optionally reducing the oxygenation there. The skilled person will appreciate that although vessel 602 is illustratively shown as a single vessel; in practice it may comprise multiple linked tanks.

A floating respirometer of the type we have described may also be employed to monitor toxicity of waste water either in a sewage treatment works or in, for example, the outfall from an industrial plant. Thus the respirometer may be provided with a supply of one or more control organisms, for example pellets of bacteria, and a mechanism to dispense these into the chamber. Such an arrangement can be used to establish known oxygen uptake rate—although since this may also be limited by the food supply optionally food may also be included with the bacteria. In one approach the respirometer is provided with a carousel of disc-shaped pellets which may be dispensed into the chamber. Then the bacteria can be culture within the chamber to determine whether the liquid sample within the chamber is toxic, potentially to determine a degree of toxicity. Such an arrangement may be used to identify undesirably high levels of contaminants such as chlorine (chlorination), the presence of one or more metals, and the presence of other toxic substances within the sample.

Although the floating respirometer we have described is particularly useful in monitoring a sewage treatment plant it may also be employed to monitor other industrial processes, in particular water-based processes. Thus, for example, embodiments of the device have been found useful in monitoring the level of bacterial contamination in water-based paint in a paint manufacturing process: such bacteria can be difficult to detect but can have significant deleterious effects on a water-based paint. The floating respirometer we have described is able to monitor the industrial process to identify when bacterial contamination is present. The skilled person will recognise that the respirometer we have described may also be employed to monitor other water-based industrial production processes in a similar manner. More generally embodiments of the respirometer may be employed to monitor other types of ‘processed waiter’—for example water in a hospital, water in an air-conditioning system and the like.

### Biomass Retention

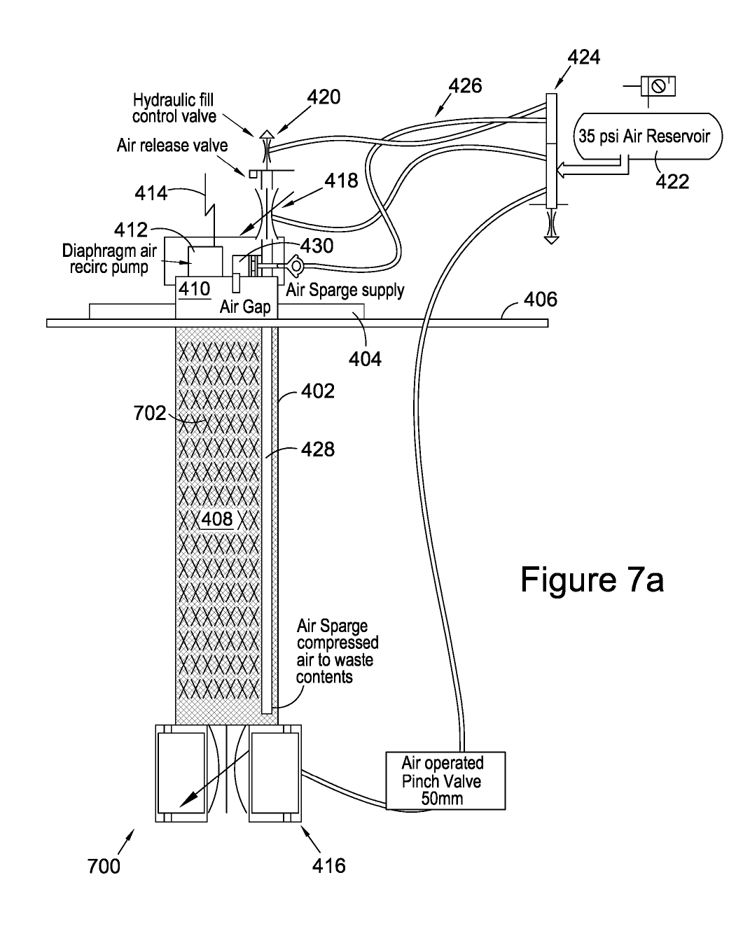

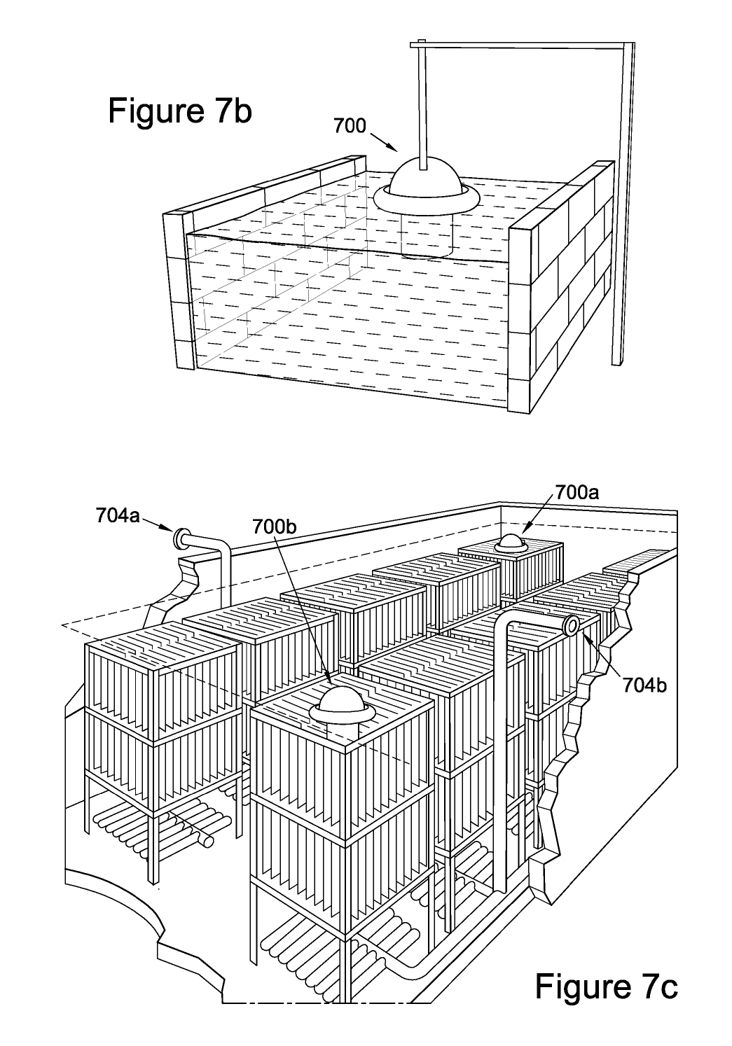

FIG. 7a shows a floating respirometer 700 in which the respirometer chamber incorporates a solid matrix 702 of large surface area on which biomass can grow. The respirometer is otherwise similar to that illustrated in FIG. 4, and like elements are indicated by like reference numerals. In one embodiment matrix 702 comprises polymer curtains or towelling such as Cleartec™ Biotextil from Cleartec™ Water Management GmbH; in another embodiment Biobeads™ from F.L.I. Water Limited, UK may be employed. Where, for example, small polypropylene curtains are employed within the sensor head these should be spaced to allow free flow of sample; where beads are employed these may be retained within the respirometer chamber. In use the biomass immobilisation solid matrix gradually becomes colonised and the respirometer reaches equilibrium with the process plant. Once equilibrium has been reached the sensor can be used to determine, for example, BOD or food to biomass ratio. Optionally the respirometer may also be used to monitor colonisation of a treatment plant as it starts up.

In some installations there may be two sensors, one at the start and one at the end of the treatment process although more sensors may be used. An entry sensor (after calibration with lab samples) may be used to measure the BOD5 of the implement and optionally from this the food to biomass ratio may be calculated. A sensor located at the exit of a treatment process will indicate the efficiency of the treatment process, in particular because this will show very little activity if the food supply has run out. The respirometer may measure head space pressure and/or may perform other measurements such as an oxygen level measurement. In general a respirometer at a sample point will indicate ‘how hard’ the immobilised biomass is having to work at that sample point. The difference in (raw or processed) signal from two (or more) respirometer sensors as described, in particular the difference between signals from an entry point and an exit point of a treatment process is indicative of the efficiency of the process and thus also of the overall ability of the process to deal with varying loads (based on the varying food supply of the influent). In addition a respirometer as described effectively mimics the waste water treatment process at the location at which It is working and may therefore be used to indicate and/or control the level or aeration, thus controlling the energy needed to maintain an optimal process. Monitoring at multiple points in a waste water treatment process enables different levels of aeration to be employed at the different locations, thus giving rise to energy savings. Thus in embodiments a waste water treatment plant may segregate treatment sections along the flow path providing separate oxygen requirement sensing and aeration control for each section. This has the potential to result in substantial energy savings.

FIG. 7b illustrates a tethered floating respirometer 700 of the type we have previously described, and FIG. 7c illustrates the use a pair of respirometers 700a, b, each monitoring a region of immobilised biomass (using curtains) with its own respective aeration 704a, b.

The skilled person will appreciate that other forms of biomass immobilisation may be employed, for example an immobilised bed or array of roughened plates. No doubt many other effective alternatives will occur to the skilled person. It will be understood that the invention is not limited to the described embodiments and encompasses modifications apparent to those skilled in the art lying within the spirit and scope of the claims appended hereto.

1. A floating respirometer, comprising:
a buoyancy device to allow the respirometer to float in an aqueous liquid;
a respirometer chamber, supported by the buoyancy device and arranged such that, when the respirometer is floating in said aqueous liquid, said chamber is partially filled with said aqueous liquid and defines an enclosed headspace above said aqueous liquid; and
a gas sensor in gaseous communication with said headspace.

2. A floating respirometer as claimed in claim 1 comprising an inlet valve beneath a water level of said chamber.

3. A floating respirometer as claimed in claim 2 wherein said inlet valve comprises a pinch valve at the bottom of the chamber.

4. A floating respirometer as claimed in claim 2 further comprising a bubble shield beneath said inlet valve.

5. A floating respirometer as claimed in claim 1, wherein said chamber further comprises a pump and conduit to pump gas from said headspace to a lower end of said chamber.

6. A floating respirometer as claimed in claim 1 further comprising at least one air release valve coupled to said headspace.

7. A floating respirometer as claimed in claim 6 further comprising an inlet valve beneath a water level of said chamber and a pneumatic control system to control operation of said inlet and air release valves to fill and empty said chamber.

8. A floating respirometer as claimed in claim 1 wherein said chamber is configured to automatically fill to a defined level set by a buoyancy of said buoyancy device.

9-10. (canceled)

11. A method of measuring a degree of respiration of living organisms in an aqueous medium, the method comprising:
providing a respiration measuring device in said aqueous medium, said respiration measuring device comprising a respiration chamber;
enabling said respiration chamber to partially fill with said aqueous medium, leaving a headspace above the aqueous medium within the device;
allowing said living organisms to respire within said respiration measuring device, in particular within said respiration chamber, such that a gaseous pressure or composition of said headspace is altered; and
measuring said alteration in said gaseous pressure or composition of said headspace to measure said degree of respiration of said living organisms.

12. A method as claimed in claim 11 further comprising inferring an oxygen demand of said living organisms from said degree of respiration.

13. A method as claimed in claim 11 further comprising inferring a degree of aeration to be provided for said aqueous medium from said degree of respiration.

14. A method as claimed in claim 11, further comprising mixing gas within said headspace with said aqueous medium sealed within said device to promote gaseous exchange to a sufficient degree that said gaseous exchange occurs at a rate faster than a rate of gas use/production by said living organisms within the device.

15. A method as claimed in claim 14 comprising promoting said gaseous exchange by pumping gas in said headspace towards the bottom of the aqueous medium in the device.

16. A method as claimed in claim 11 further comprising providing an air valve in communication with said headspace, and a pinch valve towards the bottom of the aqueous medium in the device, and controlling a sequence of operation of said valves to fill and empty said device.

17. A method as claimed in claim 16 wherein said controlling comprises controlling with a pneumatic control system.

18. A method as claimed in claim 11 wherein said headspace comprises a pressure sensor with a moveable membrane to sense a reduction in said headspace pressure.

19. A method as claimed in claim 11 further comprising arranging a chamber of said device to intersect a water line of—the device when floating such that a volume of said aqueous medium in said chamber of the device is controlled by the buoyancy of the device.

20. A method as claimed in claim 11 for continuous real time monitoring of an aqueous medium, comprising using a plurality of said respiration measuring devices and synchronising the devices such that after an initial start-up period successive devices provide successive measurements at intervals shorter than the time for which said living organisms are allowed to respire.

21-28. (canceled)

29. A floating respirometer as claimed in claim 1 further comprising a biomass growth support region within a respirometer chamber of said respirometer.

30. A method as claimed in claim 11 further comprising making a succession of measurements and retaining biomass from said measurements within a respirometer chamber of the respirometer from one measurement to the next on a biomass growth support region within a respirometer chamber of said respirometer.

31-37. (canceled)

38. A method of monitoring an aqueous medium by measuring a degree of respiration of living organisms in an aqueous medium, the method comprising:
providing a respiration measuring device in said aqueous medium, said respiration measuring device comprising a respirometer chamber;
enabling said respirometer chamber to partially fill with said aqueous medium, leaving a headspace above the aqueous medium within the device;
allowing said living organisms to respire within said respirometer chamber such that a gaseous pressure or composition of said headspace is altered; and
measuring said alteration in said gaseous pressure or composition of said headspace to measure said degree of respiration of said living organisms;
the method further comprising:
repeating said measuring on successive samples of said aqueous medium whilst retaining said living organisms within said respirometer chamber from one said sample to the next.

39. A method as claimed in claim 38 wherein said retaining comprises providing a biomass growth support region or matrix within said respiration chamber, and retaining said living organisms on said biomass growth support region or matrix.

40. (canceled)
